(19) United States
(12) Patent Application Publication
(10) Pub. No.: US 2007/0093814 A1
(43) Pub. Date: Apr. 26, 2007

(54) Dynamic spinal stabilization systems

(76) Inventors: Ronald Callahan II, Naugatuck, CT (US); Ernest Corrao, Bethel, CT (US); Stephen Maguire, Shelton, CT (US); Stephen Santangelo, New Hartford, CT (US)

Correspondence Address: McCarter & English, LLP, Financial Centre, Suite 304A, 695 East Main Street, Stamford, CT, 06901-2138, US

(21) Appl. No.: 11/247,451

(22) Filed: Oct. 11, 2005

(51) Int. Cl. A61F 2/30

(52) U.S. Cl. 606/61

(57) ABSTRACT

An elongated member forming a spinal support rod is implantable adjacent the spine of a patient, and includes an axial span or spans for spanning respective spinal levels to promote efficacious spinal support/stabilization. As with conventional spinal support rods used in connection with lumbar fusion and other related procedures, the elongated member extends in an axial direction, and is substantially dimensionally stable, both radially and axially. The elongated member is further capable of bending, flexing, and/or deflecting laterally (e.g., along any and/or substantially all transverse directions) to an extent that preserves at least some spinal motion. Such elongated members can include axial spans that manifest a radially segmented geometry relative to the axial direction, include a sleeve and a series of structural members or a coil spring enclosed within the sleeve, and/or include a coil spring and a restraining element passing at least partially through the coil spring.

### BACKGROUND

1. Technical Field

The present disclosure relates to devices, systems and methods for spinal stabilization. More particularly, the present disclosure relates to devices, systems and methods for providing dynamic stabilization to the spine via the use of elongated members spanning one or more spinal levels.

2. Background Art

Each year, over 200,000 patients undergo lumbar fusion surgery in the United States. While fusion is a well-established procedure that is effective about seventy percent of the time, there are consequences even to successful fusion procedures, including a reduced range of motion and an increased load transfer to adjacent levels of the spine, which may accelerate degeneration at those levels. Further, a significant number of back-pain patients, estimated to exceed seven million in the U.S., simply endure chronic low-back pain, rather than risk procedures that may not be appropriate or effective in alleviating their symptoms.

New treatment modalities, collectively called motion preservation devices, are currently being developed to address these limitations. Some promising therapies are in the form of nucleus, disc or facet replacements. Other motion preservation devices provide dynamic internal stabilization of the injured and/or degenerated spine, e.g., the Dynesis stabilization system (Zimmer, Inc.; Warsaw, Ind.) and the Graf Ligament. A major goal of this concept is the stabilization of the spine to prevent pain while preserving near normal spinal function.

In general, while great strides are currently being made in the development of motion preservation devices, the use of such devices is not yet widespread. One reason that this is so is the experimental nature of most such devices. For example, to the extent that a given motion preservation device diverges, whether structurally or in its method of use or implementation, from well-established existing procedures such as lumbar fusion surgery, considerable experimentation and/or testing is often necessary before such a device is given official approval by governmental regulators, and/or is accepted by the medical community as a safe and efficacious surgical option.

With the foregoing in mind, those skilled in the art will understand that a need exists for spinal stabilization devices, systems and methods that preserve spinal motion while at the same time exhibiting sufficient similarity to well-established existing spinal stabilization devices, systems and methods so as encourage quick adoption/approval of the new technology. These and other needs are satisfied by the disclosed devices, systems and methods that include elongated members for implantation across one or more levels of the spine.

### SUMMARY OF THE PRESENT DISCLOSURE

According to the present disclosure, advantageous devices, systems, kits for assembly, and/or methods for dynamic stabilization are provided. According to exemplary embodiments of the present disclosure, the disclosed devices, systems, kits and methods include an elongated member, e.g., a spinal support rod, that is configured and dimensioned for implantation adjacent the spine of a patient so as to promote efficacious spinal stabilization. The disclosed elongated member extends axially, e.g., as do spinal support rods used in connection with lumbar fusion and other related procedures. Among other similarities therewith, e.g., such as are described hereinbelow, the disclosed elongated member is substantially dimensionally stable, both radially and axially. Among some differences therewith, e.g., such as are described below, the disclosed elongate member is capable of bending, flexing, and/or deflecting laterally (e.g., along any and/or substantially all transverse directions) to an extent that preserves a degree of spinal motion.

According to exemplary embodiments of the present disclosure, the elongated member includes an axial span that extends in an axial direction across a spinal level to promote efficacious spinal stabilization thereacross, and that manifests a radially segmented geometry relative to the axial direction. In some such embodiments, the elongated member is configured and dimensioned for implantation adjacent the spine such that at least two axial spans of the elongated member extend across respective spinal levels of the spine to promote efficacious spinal stabilization across both such spinal levels. In some such embodiments, the axial span has a rod-like profile and is adapted to be coupled to the spine of the patient via attachment to conventional spine attachment devices configured for coupling conventional support rods, such as solid, relatively inflexible spinal support rods used in conjunction with spinal fusion assemblies, to the spine. In alternative embodiments of the present disclosure, the axial span is adapted to be mounted with respect to a patient's spine using alternative mounting structures/members, e.g., mounting hooks, plates, cemented stems, or the like. Such rod-like profile can include a diameter in a range of from about 5.5 mm to 6.35 mm (although alternative dimensions are contemplated), and the axial span can be adapted to permit pedicle screws to be attached to the elongated member at multiple points along the length of the axial span so as to accommodate a range of different patient anatomies and intervertebral heights. Further with respect to some such exemplary embodiments, the axial span is axially substantially rigid as against axial forces arrayed in compression and/or tension.

Still further with respect to some such exemplary embodiments, the radially segmented geometry manifested by the axial span permits the axial span to bend, flex or deflect along any and substantially all transverse directions while providing efficacious spinal stabilization across the spinal level during at least one of spinal flexion, spinal extension, spinal lateral bending, and spinal axial rotation. According to exemplary embodiments, the axial span provides efficacious spinal stabilization across the spinal level during: a) spinal flexion in which the spinal level defines an anterior bend of at least approximately five to seven degrees; b) spinal extension in which the spinal level defines a posterior bend of at least approximately three to seven degrees; and/or c) spinal bending in which said spinal level defines a lateral bend of at least approximately four to seven degrees. Yet further with respect to some such embodiments, the radially segmented geometry includes a rod of radially unitary construction and extending in the axial direction, and at least one sleeve extending in the axial direction and surrounding the rod. According to further exemplary embodiments, the rod can be fabricated, in whole or in part, from a superelastic material.

According to further embodiments of the present disclosure, a surgically implantable spinal support rod is provided that includes an axial span that extends in an axial direction so as to span at least one spinal level, wherein the axial span manifests (at least in part) a radially segmented geometry relative to the axial direction. In some such embodiments, the radially segmented geometry manifested by the axial span includes at least one pair of axially-extending adjacent surfaces adapted to move relative to each other along the axial direction during a transverse deflection of the axial span. Such at least one pair of axially-extending adjacent surfaces can include first and second substantially cylindrically shaped surfaces, wherein each such surface faces radially outerward toward the other such surface, or wherein such surfaces are substantially aligned with respect to each other. In others of such embodiments, the axial span has a rod-like profile, and is adapted to be coupled to the spine of the patient via attachment to conventional spine attachment devices configured for coupling conventional support rods to the spine for purposes of spinal fusion. Such rod-like profile of the axial span can include a diameter in a range of from about 5.5 mm to 6.35 mm, although alternative dimensions and/or dimensional ranges may be employed.

In accordance with still further embodiments of the present disclosure, a kit for assembling a dynamic spinal support system is provided. Such kit includes a spinal support rod having an axial span extending in an axial direction so as to span at least one spinal level, and manifesting a radially segmented geometry relative to said axial direction. Such kit also includes a plurality of spine attachment devices attachable to the axial span so as to couple the spinal support rod to the spine of the patient across the spinal level. In some such embodiments, at least one of such spine attachment devices includes a pedicle screw, hook, plate and/or cemented stem.

In accordance with another embodiment of the present disclosure, the elongated member includes an axial span that extends in an axial direction across at least one spinal level to promote efficacious spinal stabilization thereacross, and that includes a sleeve and a series of structural members aligned along the axial direction, enclosed within the sleeve, and adapted to support the sleeve against lateral buckling, e.g., when the sleeve experiences a lateral bend and is supporting the spine across the at least one spinal level. In some such embodiments, the sleeve is adapted to generate an internal spring force in opposition to the lateral bend as the sleeve deflects so as to accommodate and moderate the lateral bend. In exemplary embodiments, the sleeve can be fabricated, at least in part, from a superelastic material, such as an alloy of nickel titanium. The structural members can be substantially spherical in shape and, in such embodiments, the sleeve can be substantially cylindrical in shape.

In accordance with yet another embodiment of the present disclosure, the elongated member includes an axial span that extends in an axial direction across at least one spinal level to promote efficacious spinal stabilization thereacross, and further includes an axial sleeve and a coil spring disposed within the axial sleeve. In some such embodiments, the sleeve is fabricated from a superelastic material and/or an alloy of titanium. In some other such embodiments, the sleeve is fabricated from a polymeric material. In some other such embodiments, the coil spring is sized and oriented so as to support a peripheral shape of the axial sleeve against at least one of crushing and buckling during spinal stabilization.

In accordance with another embodiment of the present disclosure, the elongated member includes an axial span that extends in an axial direction across at least one spinal level to promote efficacious spinal stabilization thereacross, and further includes an axially-extending coil spring and a restraining element disposed within the coil spring and extending at least partially through the coil spring in the axial direction so as to limit an axial extension of the elongated member. In some such embodiments, the restraining element includes a cable adapted to render the elongated member substantially rigid as against axial forces arrayed in compression. The cable can take the form of a wire rope cable.

According to further embodiments of the present disclosure, a surgically implantable spinal support rod is provided that includes an axial span that extends in an axial direction so as to span at least one spinal level, wherein the disclosed spinal support bar is of unitary construction, both along the axial direction, and radially relative to the axial direction, and is further adapted to deflect laterally so as to permit at least three to seven degrees of bending in the spine across the at least one spinal level in at least one of spinal flexion, spinal extension, and spinal lateral bending. In some such embodiments, the spinal support bar manifests a substantially constant cross-sectional geometry across the at least one spinal level, e.g., a circular cross-sectional geometry. In some other such embodiments, the spinal support bar includes a central span extending in the axial direction, and channels formed in the central span so as to increase transverse flexibility of the central span. Such channels can extend in the axial direction, and/or such channels can extend transversely relative to the axial direction. In some other such embodiments, the spinal support bar includes a central span, a first end span, and a second end span disposed opposite the central span from the first end span. The central span may be associated with a reduced cross-sectional area relative to respective cross-sections of the first and second end spans, e.g., the central span can be associated with a circular cross section of a reduced diameter relative to respective circular cross-sections of the first and second end spans.

The elongated members/spinal support rods of the present disclosure, and/or the spinal stabilization devices/systems of the present disclosure incorporating such elongated members/spinal support rods, advantageously include one or more of the following structural and/or functional attributes:
Spine surgery patients whose conditions indicate that they would benefit from retaining at least some spinal motion in flexion, extension, and/or axial rotation may be fitted with a dynamic spinal stabilization device/system as disclosed herein rather than undergo procedures involving substantial immobilization as between adjacent vertebrae;
The elongated members/spinal support rods in accordance with the present disclosure are compatible (e.g., by virtue of standard diameter sizing, substantial dimensional/diametrical stability, and/or rigidity in axial tension and axial compression, etc.) with most rod attachment hardware presently being implanted in conjunction with lumbar fusion surgery, enhancing the likelihood of quick adoption by the medical community and/or governmental regulatory approval;
The elongated members/spinal support rods disclosed herein are adaptable to pedicle screw, hook, plate and/or stem attachment, can be used across one or more spinal levels; permit at least approximately seven degrees of spinal extension, spinal flexion, and/or spinal lateral bending as between adjacent spinal vertebrae, and allow for adjustable attachment points along their axial lengths to accommodate differing patient anatomies.

Advantageous spine stabilization devices, systems, kits for assembling such devices or systems, and methods may incorporate one or more of the foregoing structural or functional attributes. Thus, it is contemplated that a system, device, kit and/or method may utilize only one of the advantageous structures/functions set forth above, or all of the foregoing structures/functions, without departing from the spirit or scope of the present disclosure. Stated differently, each of the structures and functions described herein is believed to offer benefits, e.g., clinical advantages to clinicians or patients, whether used alone or in combination with others of the disclosed structures/functions.

Additional advantageous features and functions associated with the devices, systems, kits and methods of the present disclosure will be apparent to persons skilled in the art from the detailed description which follows, particularly when read in conjunction with the figures appended hereto. Such additional features and functions, including the structural and mechanistic characteristics associated therewith, are expressly encompassed within the scope of the present invention.

### BRIEF DESCRIPTION OF THE DRAWINGS

To assist those of ordinary skill in the art in making and using the disclosed devices and systems, reference is made to the appended figures, in which:

FIGS. 1, 2 and 3 are respective side, top, and end views of a dynamic spinal stabilization device/system implanted into the spine of a patient, in accordance with a first embodiment of the present disclosure;

FIG. 4 is a downward perspective view of an elongated member of the spinal stabilization device/system of FIGS. 1-3, at least a portion of the internal structure of which is illustrated via a partial cutaway;

FIG. 5 is a side illustration of the elongated member of FIG. 4;

FIG. 6 is a cross-sectional view of the elongated member of FIGS. 4 and 5, taken along section line 6-6 in FIG. 5;

FIG. 7 is a side illustration of the spinal stabilization device/system of FIGS. 1-3, wherein the patient is in spinal flexion;

FIG. 8 is a side illustration of the spinal stabilization device/system of FIGS. 1-3, wherein the patient is in spinal extension;

FIGS. 9 and 10 are top views of the spinal stabilization device/system of FIGS. 1-3, wherein the spine of the patient is bending along the left and right lateral directions, respectively; and

FIGS. 11 and 12 are end views of the spinal stabilization device/system of FIGS. 1-3, wherein the spine of the patient is subject to axial rotation to the right and to the left, respectively;

FIGS. 13-20, 22 and 25 are downward perspective view of elongated members which may be substituted for the elongated member of FIGS. 4-6 in accordance with respective modifications and/or alternative embodiments of the spinal stabilization/system of FIGS. 1-3;

FIG. 21 is a cross-sectional view of the elongated member of FIG. 20;

FIGS. 23-24 are cross-sectional views of the elongated member of FIG. 22; and

FIGS. 26-27 are cross-sectional views of the elongated member of FIG. 25.

### DESCRIPTION OF EXEMPLARY EMBODIMENTS

The present disclosure provides advantageous devices, systems and methods for providing dynamic spinal stabilization. More particularly, the present disclosure provides elongated members in the form of rods that are suitable for surgical implantation across multiple spinal levels for purposes of support and stabilization in flexion, extension and/or axial rotation, and that are also laterally flexible so as to provide a range of motion in spinal flexion, extension and/or axial rotation.

The exemplary embodiments disclosed herein are illustrative of the advantageous spinal stabilization devices/systems and surgical implants of the present disclosure, and of methods/techniques for implementation thereof It should be understood, however, that the disclosed embodiments are merely exemplary of the present invention, which may be embodied in various forms. Therefore, the details disclosed herein with reference to exemplary dynamic stabilization systems and associated methods/techniques of assembly and use are not to be interpreted as limiting, but merely as the basis for teaching one skilled in the art how to make and use the advantageous spinal stabilization systems and alternative surgical implants of the present disclosure.

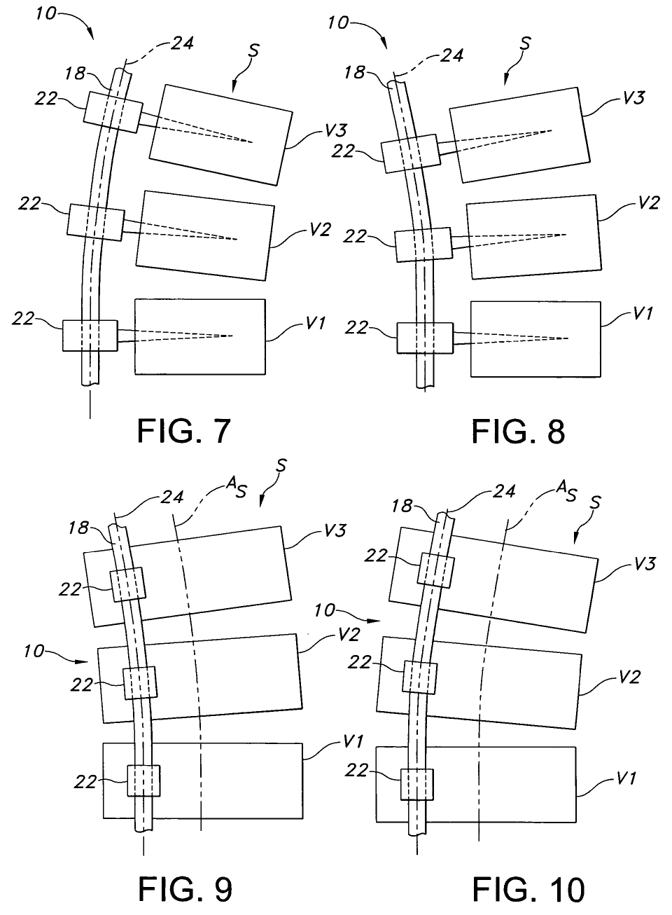

With reference to FIGS. 1-3, a dynamic spinal stabilization system 10 is shown implanted into and/or relative to the spine S of a patient, such spine S being rendered schematically in FIGS. 1-3 (as well as in FIGS. 7-12, the details of which are described more fully hereinbelow) in the form of three adjacent sequential vertebrae V1, V2 and V3 separated by corresponding intervertebral gaps G1 and G2. The dynamic stabilization system 10 is attached to the spine S along one lateral side thereof as defined by a bilateral axis of symmetry As thereof (another dynamic spine stabilization system 10 (not shown) can be attached to the spine S along the other lateral side thereof as desired and/or as necessary). The spinal stabilization system 10 includes three spine attachment elements 12, 14, 16, and an elongated member 18 spanning all of the vertebrae V1, V2, V3 (e.g., at least insofar as the gaps G1, G2 therebetween).

Each of the spine attachment elements 12, 14, 16 of the spinal stabilization system 10 includes an attachment extension 20 (depicted at least partially schematically) and an attachment member 22 (also depicted at least partially schematically). The spine attachment elements 12, 14, 16 are securely affixed to the respective vertebrae V1, V2, V3 via respective ends of the attachment extensions 20 being embedded within corresponding voids in the tissue of the respective vertebrae V1, V2, V2, and being securely retained therein (i.e., so as to prevent the attachment extensions 20 from being pulled out of their respective voids, or rotated with respect thereto, whether axially or otherwise). The attachment extensions 20 are embedded into and/or retained within their respective vertebral voids via suitable conventional means, such as a helical thread and/or a helically-shaped inclined plane formed on the respective attachment extension 20, a biocompatible adhesive, or other means of embedding and/or retention. The attachment extensions 20 form respective parts of and/or are mounted with respect to respective pedicle screws of conventional structure and function in accordance with at least some embodiments of the present disclosure. The attachment extensions 20 form parts of other types of structures than that of conventional pedicle screws in accordance with some other embodiments of the present disclosure, e.g., hooks, plates, stems or the like.

The attachment extensions 20 and attachment members 22 of the spine attachment elements 12, 14, 16 are attached or coupled with respect to each other at respective ends of the attachment extensions 20 opposite the ends thereof that are embedded within the tissue of the respective vertebrae V1, V2, V3. Movable joints are advantageously formed at the points where the attachment extensions 20 and the attachment members 22 are attached/coupled. In at least some embodiments of the present disclosure, the ends of the attachment extensions 20 that are attached/coupled with respect to the respective attachment members 22 include respective pedicle screw heads of conventional structure and function. In some other embodiments of the present disclosure, such ends include types of structure other than that of conventional pedicle screw heads. The movable joints formed between the attachment extensions 20 and the attachment members 22 may advantageously permit relatively unconstrained relative rotation (e.g., global rotation) therebetween, as well as at least some rotation of each attachment member 22 about an axis defined by the corresponding attachment extension 20. The structure and function of the movable joints between the attachment extensions 20 and the attachment members 22 of the respective spine attachment elements 12, 14, 16 will be described in greater detail hereinafter.

The attachment members 22 of the spine attachment elements 12, 14, 16 are generally configured and dimensioned so as to be operatively coupled to known spinal support rods (not shown) such as spinal support rods of conventional structure and having a standard diameter (e.g., from about 5.5 mm to about 6.35 mm, although alternative dimensions may be employed) and that are commonly used in connection with lumbar fusion surgery and/or other spinal stabilization procedures. For example, in accordance with some embodiments of the present disclosure, each of the attachment members 22 is configured to couple to a conventional spinal support rod (not shown) so as to prevent relative movement between the attachment members 22 and the rod in a direction transverse (e.g., perpendicular) to the rod's axial direction of extension, and at least one of the attachment members 22 is further adapted to prevent relative movement between such attachment member 22 and the rod along the rod's axial direction of extension. The particular structures and characteristic functions of the attachment members 22 of the spine attachment elements 12, 14, 16 are discussed in greater detail hereinafter.

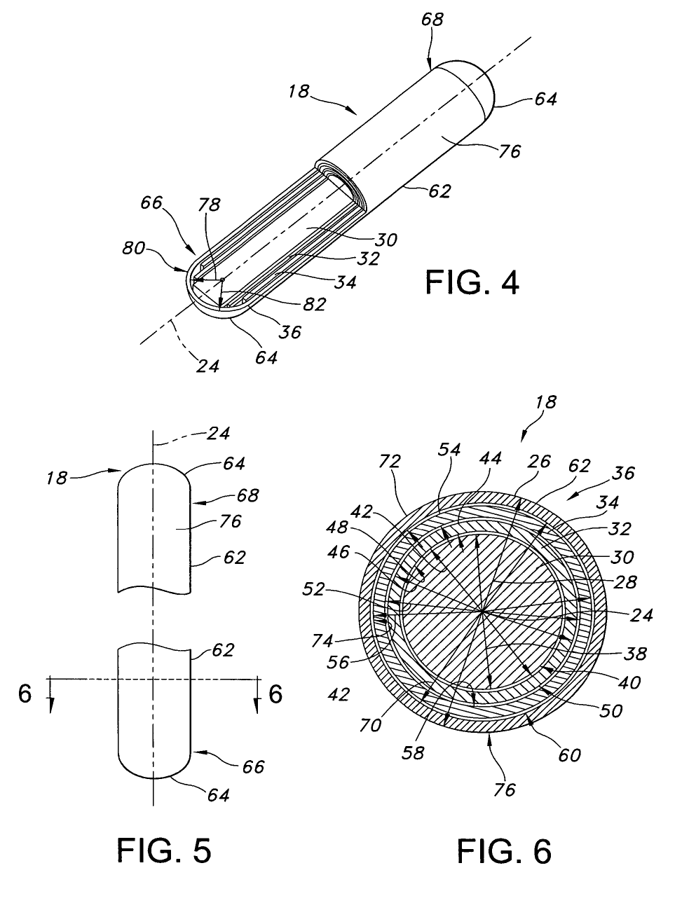

Referring now to FIGS. 4-6, the exemplary elongated member 18 of the spinal stabilization system 10 (FIG. 1) includes an axis 24 defined by an axial/longitudinal direction along which the elongated member 18 characteristically extends. As shown in FIG. 6, exemplary elongated member 18 has an outer perimeter 26 in end view that has a substantially circular shape. The circular outer perimeter 26 defines a basic diameter 28 of the elongated member 18 of an extent that is typically consistent with that of conventional spinal stabilization rods (e.g., an extent in a range of from about 5.5 mm to about 6.35 mm or alternative dimension) such that the elongated member 18 is compatible with hardware designed to couple to conventional spinal stabilization rods and associated anatomical features and criteria. Accordingly, and referring again to FIGS. 1-3, the elongated member 18 is generally compatible with the spine attachment elements 12, 14, 16. More particularly, the elongated member 18 is coupled to the attachment members 22 of the spine attachment elements 12, 14, 16 such that transverse movement of the elongated member 18 relative to the respective attachment members 22 is substantially limited and/or prevented. This is consistent with the support and stabilization functions (described in greater detail hereinafter) of the elongated member 18 with respect to the spine S.

With respect to at least one of the attachment members 22, the elongated member 18 is coupled thereto such that motion/translation of the elongated member 18 in the axial direction (i.e., in the direction of the axis 24) relative to such attachment member(s) is substantially limited and/or prevented. This ensures that the elongated member 18 is prevented from freely and/or uncontrollably moving/translating in the axial direction with respect to the spine attachment elements 12, 14, 16 in the context of the overall spinal stabilization system 10. Moreover, in accordance with the embodiment of the present disclosure illustrated in FIGS. 1-6, the global joints formed between the attachment members 22 and the attachment extensions 20 of the respective spine attachment elements 12, 14, 16 generally allow the attachment members 22 to rotate to some degree along with the elongated member 18 relative to the spine S. The significance of such aspects of the connection between the elongated member 18 and the spine attachment elements 12, 14, 16 is described more fully hereinafter.

The elongated member 18 is also similar to conventional spinal stabilization rods in that it is substantially dimensionally stable in the radial direction (e.g., transversely/perpendicularly relative to the axial direction of extension of the elongated member 18 as represented by the axis 24). Accordingly, the elongated member 18 is capable of withstanding radially-directed compression forces imposed by any and/or all of the attachment members 22 either during the process of implanting the elongated member 18 along the spine S (e.g., in response to clamping forces imposed by any attachment member 22 on the elongated member 18) or during in situ use of the spinal stabilization system 10 (the details of such use being described more fully hereinafter). In accordance with at least some embodiments of the present disclosure, the material and structural aspects of the elongated member 18 described herein render the elongated member 18 substantially rigid in axial tension, as well as substantially incompressible when subjected to axially-directed compression forces.

Still referring to FIGS. 4-6, exemplary elongated member 18 includes four axially-extending structures, to wit: a rod 30, a first inner sleeve 32 surrounding the rod 30, a second inner sleeve 34 surrounding the first inner sleeve 32, and an outer sleeve 36 surrounding and/or enveloping the outer sleeve 34. The rod 30 has a substantially circular cross-section defined by a basic diameter 38 that has an extent of approximately 2.0 to 3.0 mm, and that is substantially constant along the axial length of the elongated member 18 (e.g., along the axis 24). Accordingly, and at least when the rod 30 is in a straight and/or linear configuration, a peripheral outer surface 40 of the rod 30 is substantially cylindrical. The first inner sleeve 32 is also substantially circular in cross-section, being characterized by a substantially axially constant inner diameter 42 accommodative of the basic diameter 38 of the rod 30, a radial thickness 44, and a substantially axially constant outer diameter 46. At least when the inner sleeve 32 is in a straight and/or linear configuration, both an inner surface 48 and a peripheral outer surface 50 of the first inner sleeve 32 are substantially cylindrical. The second inner sleeve 34 is also substantially circular in cross-section, being characterized by a substantially axially constant inner diameter 52 accommodative of the outer diameter 46 of the first inner sleeve 32, a radial thickness 54, and a substantially axially constant outer diameter 56. At least when the inner sleeve 32 is in a straight and/or linear configuration, both an inner surface 58 and a peripheral outer surface 60 of the second inner sleeve 34 are substantially cylindrical.

The outer sleeve 36 includes an axial portion 62 and two end caps 64 disposed on opposite ends 66, 68 of the axial portion 62 from each other. The axial portion 62 is substantially circular in cross-section, being characterized by a substantially axially constant inner diameter 70 accommodative of the outer diameter 56 of the second inner sleeve 34, a radial thickness 72, and the outer diameter 28, which is further substantially axially constant. At least when the axial portion 62 is in a straight and/or linear configuration, both an inner surface 74 and a peripheral outer surface 76 of the axial portion 62 are substantially cylindrical. The end caps 64 are substantially hemispherical in shape, being characterized by a substantially constant inner radius 78, a radial thickness 80, and a substantially constant outer radius 82 that is of an extent complementary to that of the outer diameter 74 of the axial portion 62.

In at least some embodiments of the present disclosure, including the embodiment schematically depicted herein, the rod 30, the first and second inner sleeves 32, 34, and the outer sleeve 36 are each fabricated from a superelastic material, e.g., such as a nickel titanium alloy. The significance of such material compositions of these components is described more fully hereinbelow.

The rod 30 extends substantially the entire length of the elongated member 18 along the axis 24, beyond the ends 66, 68 of the axial portion 62 of the outer sleeve 36, and into the interior spaces defined by the end caps 64 thereof, e.g., substantially as far as the inner wall surfaces thereof. The rod 30 is also of unitary construction throughout its length and cross-section. Combined with the inherently compact circular shape of the rod 30 in cross section, the superelastic material composition and unitary construction of the rod 30 render it substantially radially incompressible. The first and second inner sleeves 32, 34 extend substantially the full axial distance between the inner wall surfaces of the end caps 64, being only slightly shorter than the rod 30 so as to accommodate the respective radiused geometries of the end caps 64. The cumulative transverse extent of the diameter 38 of the rod 30, the radial thickness 44 of the first inner sleeve 32, and the radial thickness 54 of the second inner sleeve 34, represents a substantial proportion of the transverse extent of the inner diameter 60 of the axial portion 62 of the outer sleeve 36. More particularly, the radial/peripheral spaces between the rod 30 and the first inner sleeve 32, and/or between the first and second inner sleeves 32, 34, are relatively small. At the same time, the above-described coordination among the various diameters of the axially-extending structures of the elongated member 18 is also designed so as to reduce and/or eliminate any undue interference (e.g., via friction or otherwise) with the flexure-related functions of the elongated member 18, which functions are described more fully hereinbelow.

At least in part because of the closely matched diametrical dimensions of the rod 30 and the first inner sleeve 32, the rod 30 substantially fully supports the first inner sleeve 32 against crushing, buckling, and/or plastic deformation during bending, flexure, and/or deflection of the overall elongated member 18 (e.g., during in situ use and/or during representative mechanical testing). For example, in accordance with at least some embodiments of the present disclosure, the attachment members 22 associated with the spine attachment elements 12, 14, 16 apply radial compression, radial impingement, and/or clamping forces to the elongated member 18 at their respective points of contact therewith, and the rod 30 provides structural and/or shape support to the first inner sleeve 32 at, along, and/or adjacent to such points of contact. The first inner sleeve 32, being substantially fully supported against undue radial deflection or deformation (see above), provides similar structural and/or shape support to the second inner sleeve 34. So, in turn, does the second inner sleeve 34 provide structural and/or shape support to the axial portion 62 of the outer sleeve 36. Accordingly, the overall elongated member 18 is substantially radially incompressible along its entire axial length (e.g., along the axis 24), e.g., as against such bending stresses, radial impingement, and/or clamping or other transverse/radial forces as are applied to the elongated member 18, whether by the attachment members 22, or otherwise.

In operation, e.g., when incorporated in the spinal stabilization system 10 adjacent the spine S of a patient as described hereinabove, the elongated member 18 is capable of supporting the spine S in any one or more, or all, of spinal flexion, spinal extension, and axial rotation. As may be seen by comparing FIGS. 1 and 7, the elongated member 18 of the spinal stabilization system 10 is sufficiently flexible to bend from a substantially linear configuration (FIG. 1) to a configuration in which the elongated member 18 includes an anterior bend (FIG. 7), while being also sufficiently stiff to provide ample support to the vertebrae V1, V2, V3 of the spine S against undue spinal flexion, as determined by the anatomy and/or particular medical condition of the patient. In accordance with some embodiments of the present disclosure, the elongated member 18 is dimensioned and configured so as to permit such spinal flexion between adjacent vertebrae (e.g., between vertebrae V1 and V2, or between vertebrae V2 and V3) to an extent of at least approximately three to seven degrees.

As may be seen by comparing FIGS. 1 and 8, the elongated member 18 of the spinal stabilization system 10 is sufficiently flexible to bend from a substantially linear configuration (FIG. 1) to a configuration in which the elongated member 18 includes a posterior bend (FIG. 8), while being also sufficiently stiff to provide ample support to the vertebrae V1, V2, V3 of the spine S against undue spinal extension, as determined by the anatomy and/or particular medical condition of the patient. In accordance with some embodiments of the present disclosure, the elongated member 18 is dimensioned and configured so as to permit such spinal extension between adjacent vertebrae (e.g., between vertebrae V1 and V2, or between vertebrae V2 and V3) to an extent of at least approximately three to seven degrees.

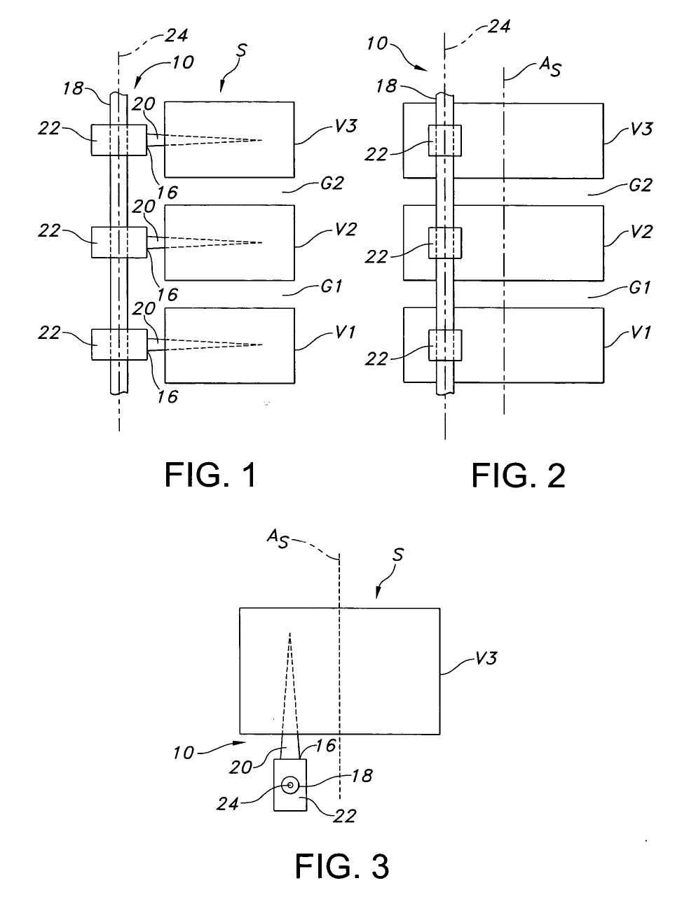

As may be seen by comparing FIG. 2 to FIGS. 9 and 10, respectively, the elongated member 18 of the spinal stabilization system 10 is sufficiently flexible to bend from a substantially linear configuration (FIG. 2) to a configuration in which the elongated member 18 includes a leftward lateral bend (FIG. 9) or a rightward lateral bend (FIG. 10), as reflected in the respective curves in the axis of symmetry A_{S} of the spine S, while being also sufficiently stiff to provide ample support to the vertebrae V1, V2, V3 of the spine S against undue spinal lateral bending, as determined by the anatomy and/or particular medical condition of the patient. In accordance with some embodiments of the present disclosure, the elongated member 18 is dimensioned and configured so as to permit such spinal lateral bending between adjacent vertebrae (e.g., between vertebrae V1 and V2, or between vertebrae V2 and V3) to an extent of at least approximately three to seven degrees.

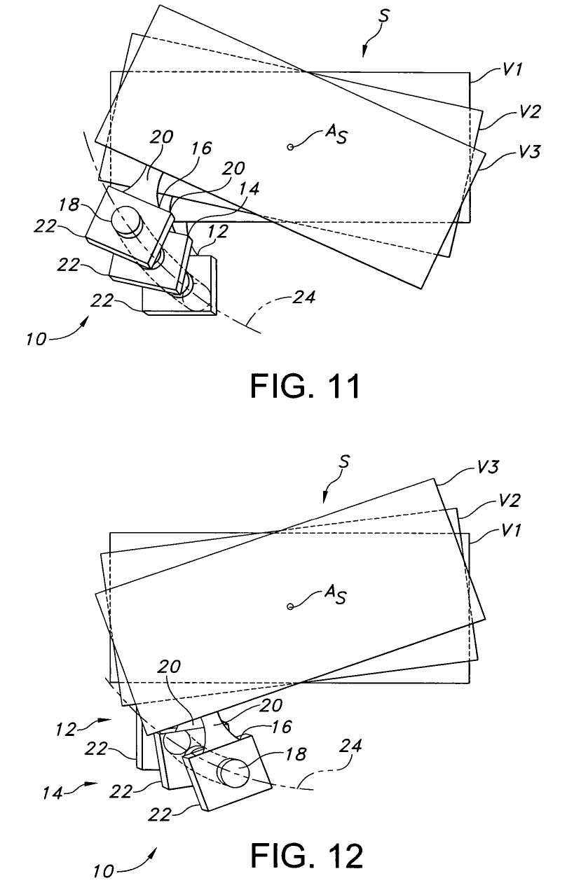

As may be seen by comparing FIG. 3 to FIGS. 11 and 12, respectively, the elongated member 18 of the spinal stabilization system 10 is sufficiently flexible to bend from a substantially linear configuration (FIG. 3) to a configuration in which the elongated member 18 includes a leftward helical bend (FIG. 11) or a rightward helical bend (FIG. 12) about the axis of symmetry A_{S} of the spine S, while being also sufficiently stiff to provide ample support to the vertebrae V1, V2, V3 of the spine S against undue spinal twist/axial rotation, as determined by the anatomy and/or particular medical condition of the patient. In accordance with some embodiments of the present disclosure, the elongated member 18 is dimensioned and configured so as to permit such axial rotation between adjacent vertebrae (e.g., between vertebrae V1 and V2, or between vertebrae V2 and V3) to an extent of at least approximately four (4) degrees. As is particularly evident in the illustrations provided in FIGS. 11 and 12, the joints between the attachment members 22 and the attachment extensions 20 of the spine attachment elements 12, 14, 16 permit the attachment members 22 ranges of motion relative to the respective attachment extensions 20, and relative to each other, sufficient to track even a complex helical bend, free from undue friction and/or binding.

Further with reference to each of FIGS. 7-12, the relationship between the attachment members 22 and the elongated member 18 during the formation and/or relaxation of bends in the elongated member 18 is such as to permit and/or restrict relative axial/longitudinal relative movement between the attachment members 22 and the elongated member 18 along the axial direction of extension of the elongated member 18 (e.g., along the axis 24), as needed or as desired (e.g., depending on the desired function or functions of the spinal stabilization system 10, the needs of the particular patient, and/or the length of the elongated member 18, among other considerations).

Referring still further to FIGS. 4-6, the elongated member 18 is configured to permit relative movement as between respective adjacent surfaces of its axially-extending structures. More particularly, at least axially-directed relative movement is respectively permitted as between: 1) the peripheral outer surface 40 of the rod 30 and the inner surface 48 of the first inner sleeve 32; 2) the peripheral outer surface 50 of the first inner sleeve 32 and the inner surface 58 of the second inner sleeve 34; and 3) the peripheral outer surface 60 of the first inner sleeve 32 and the inner surface 74 of the axial portion 62 of the outer sleeve 36. As relates to the operation of the spinal stabilization system 10 shown and described above with reference to FIGS. 7-12, transverse bending, flexure, and/or deflection of the elongated member 18 (e.g., as is produced during spinal flexion, extension and/or axial rotation) will generally result in at least some axially-directed relative movement as between the above-mentioned pairs of radially-adjacent, axially-extending surfaces. Such movement between internal surfaces tends to dissipate, reduce and/or prevent internal stresses from accumulating at corresponding radial intervals within the elongated member 18. As those of skill in the art will recognize in light of the present disclosure, the dissipation and/or exclusion of such internal stresses via axially-directed relative motion between such pairs of radially-adjacent, axially extending surfaces renders the elongated member 18 more flexible, e.g., to at least a certain extent, than that which would otherwise be the case. For example, as compared to an elongated member (not shown) having the same outer diameter as the elongated member 18, and being fabricated from the same superelastic material thereof, but having a unitary (e.g., rather than multicomponent) construction along the radial direction, the elongated member 18 offers less resistance, e.g., to at least a certain extent, to transverse bending, flexure and/or axial rotation.

It should be appreciated that numerous advantages are provided by the elongated member 18 and/or by devices such as the spinal stabilization device 10 that incorporate the elongated member 18 in accordance with the foregoing description to provide dynamic stabilization to the spine of a patient. Spine surgery patients whose conditions indicate that they would benefit from retaining at least some spinal motion in flexion, extension and/or axial rotation may benefit through implantation of the dynamic spinal stabilization device 10 rather than undergoing procedures involving substantial immobilization as between adjacent vertebrae. The elongated member 18 (e.g., by virtue of its standard diameter sizing, substantial dimensional stability, and rigidity in tension and/or compression) is compatible with most rod attachment hardware presently being implanted in conjunction with lumbar fusion surgery and other spinal procedures, providing at least some basic similarity between the spinal stabilization device 10 and existing spinal stabilization devices. Such similarity is advantageous insofar as it tends to simplify the process of seeking widespread industry acceptance and/or regulatory approval. Exemplary embodiments of elongated member 18 are adaptable to pedicle screw attachment or other mounting systems (e.g., hooks, plates, stems and the like), allow for use across two or more spinal levels, permit at least approximately three to seven degrees of lateral flexibility in spinal extension, spinal flexion, and/or spinal lateral bending as between adjacent spinal vertebrae, and allow for adjustable pedicle screw attachment points along the elongated member 18 to accommodate differing patient anatomies.

The axially symmetrical structure of the elongated member 18 affords an even, predictable level of bending flexibility (or, conversely, bending stiffness) in all lateral directions to facilitate smooth bending, and defines a substantial outer diameter compatible with the same conventional spine attachment hardware normally used in conjunction with solid, substantially laterally inflexible support rods. At the same time, the elongated member 18 is substantially radially incompressible, such that it maintains an adequate degree of rigidity against axial forces in compression (as well as in tension) for purposes of spinal support/stabilization. The peripheral outer surface 76 of the elongated member 18 has a regular cylindrical shape, facilitating secure coupling with hardware designed for coupling to cylindrically-shaped support rods of full diameter and substantially unitary structure. The superelastic material from which the different axially-extending components of the elongated member 18 may be fabricated (at least in part) resists buckling, distension, elastic deformation, and/or galling, and has excellent memory such that the bends produced in the elongated member 18 will be substantially fully removed in the event outside forces acting upon the elongated member are eliminated. Full encapsulation of all other axially-extending components of the elongated member 18 within the axial portion 62 and the end caps 64 of the outer sleeve 36 reduces and/or eliminates the risk that particulate matter, e.g., from metal-metal interaction, will be released in situ. The outer sleeve 36, being fabricated from a superelastic material, includes an inherent degree of stiffness against bending, at least to the extent that its cylindrical shape is supported and/or preserved during bending, flexure, and/or deflection of the elongated member 18. Accordingly, the radial thickness 72 of the axial portion 62 of the outer sleeve 36 can be pre-selected based on that proportion of the bending stiffness of the elongated member 18 which is intended to be supplied by the outer sleeve 36 itself.

It should also be noted that the elongated member 18, and/or the dynamic spinal stabilization device 10 of which the elongated member 18 forms a part, are subject to numerous modifications and/or variations. For example, the elongated member 18 can be attached in many different ways to the attachment members 22 of the respective spine attachment elements 12, 14, 16, including embodiments wherein at least one of the attachment members 22 includes an axial hole through which the elongated member 18 either extends freely in the axial direction, or is clamped in place so as to prevent relative axial motion/translation, and embodiments wherein at least one of the attachment members 22 forms a hook-like structure that includes no clamping means and therefore does not limit axial relative motion/translation of the elongated member 18. Many other variations in the spine attachment elements 12, 14, 16 are also possible, including the number of same provided in the context of the spinal stabilization device 10 (e.g., only two, four or more, etc.), as well as the method by which any or all are attached to their respective spinal vertebrae. The elongated member 18 can accordingly be shortened or lengthened, so as to be suitable for spanning a single pair of adjacent vertebrae, or more than three adjacent vertebrae. The number of inner sleeves can be only one, or more than two, and the diameters thereof, and/or of the rod 30, can be changed as necessary, and/or as desired, e.g., so as to produce a particular (e.g., predefined) amount of bending stiffness in the elongated member 18.

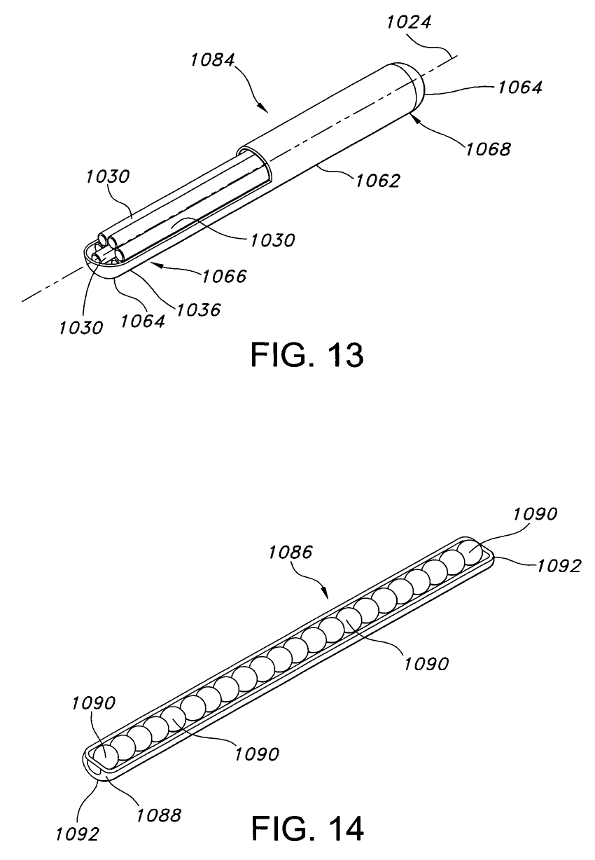

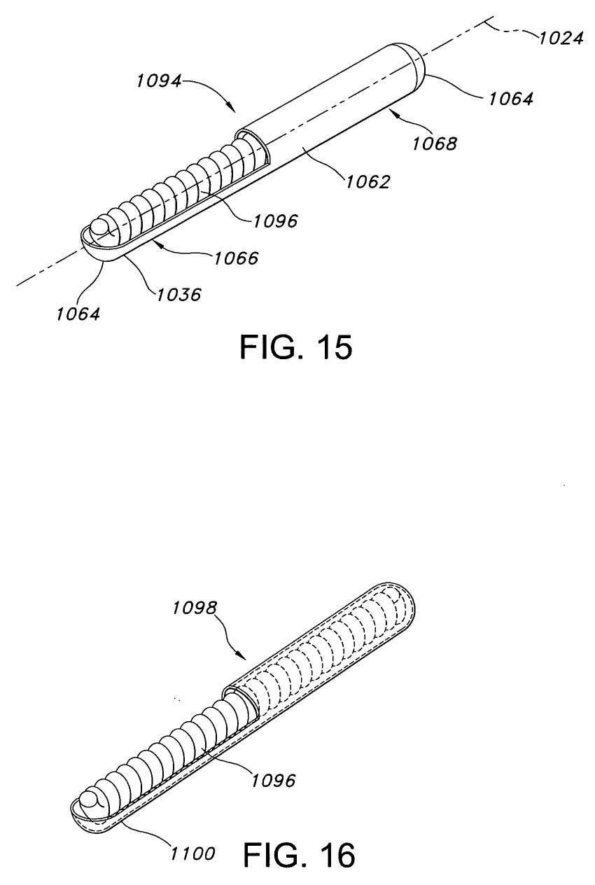

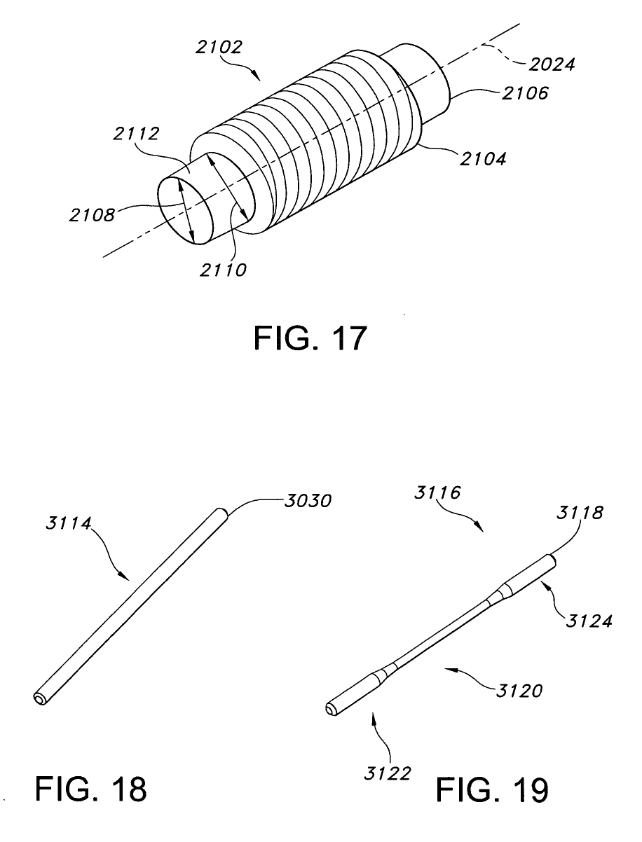

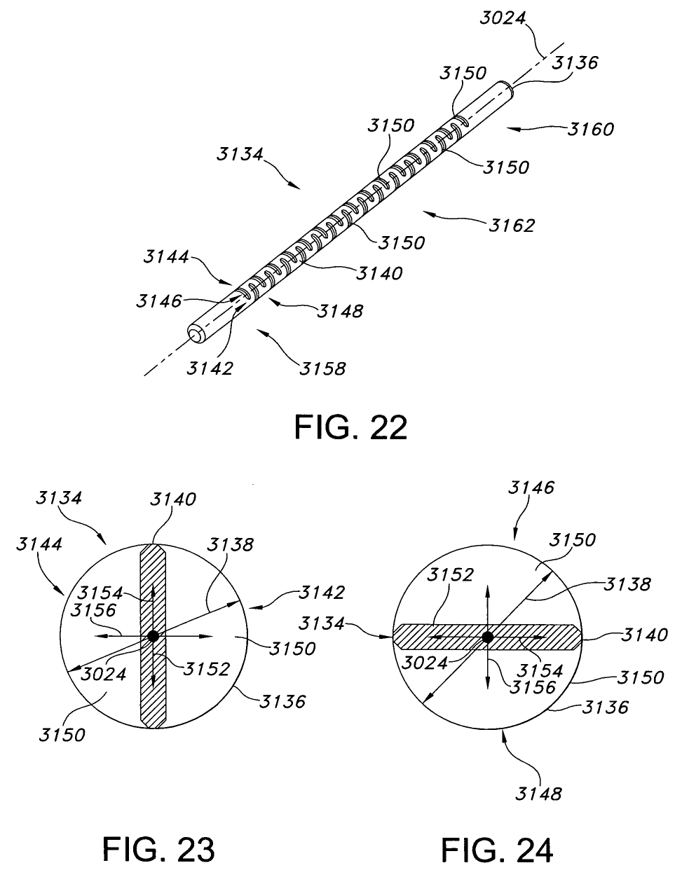

The spinal stabilization system 10 of FIGS. 1-3 and 7-12 is subject to further modification, e.g., via replacement therein of the elongated member 18 of FIGS. 4-6 with elongated members exhibiting certain differences, such as differences in configurations, structures, materials, properties and/or features, relative to the elongated member 18, as well as certain similarities with respect thereto. More particularly, FIGS. 13-15 illustrate elongated members which are similar to the elongated member 18 at least insofar as they incorporate a similar outer sleeve and include more than one axially-extending component, but which also include differences at least as described below. FIG. 16 illustrates an elongated member that is similar to the elongated member of FIG. 15 at least insofar as it incorporates a geometrically similar outer sleeve, but which also includes differences in its outer sleeve at least as described below. FIG. 17 illustrates an elongated member that is similar to the elongated member 18 at least insofar as it includes more than one axially-extending component, but which also includes differences at least as described below. FIGS. 18-21 illustrate elongated members that are similar to the elongated member at least insofar as they include axially-extending bars fabricated (at least in part) from a superelastic material, but which also include differences at least as described below. FIGS. 22-24 and 25-27 illustrate respective elongated members that are similar to the elongated member 18 at least insofar as they are flexible in more than one lateral/transverse direction, but which also include differences at least as described below. Other elongated members can similarly be substituted for the elongated member 18 in accordance with the present disclosure.

Elements illustrated in FIGS. 13-27 which correspond substantially to the elements described above with reference to FIGS. 1-12, and/or to elements illustrated previously with respect to another of FIGS. 13-27, have been designated with corresponding reference numerals increased by one or more increments of one thousand. The elongated members shown in FIGS. 13-27 operate and are constructed in manners consistent with the foregoing description of the elongated member 18, unless it is stated otherwise. In addition, the elongated members shown in FIGS. 13-17 feature the same advantages as are described hereinabove with respect to the elongated members, and are subject to the same type and degree of variations and/or modifications, unless it is stated otherwise, or unless a contrary conclusion is required based on the corresponding descriptions and/or illustrations.

Turning now to FIG. 13, an elongated member 1084 is illustrated that includes an outer sleeve 1036, and an arrangement of rods 1030 (e.g., seven are shown of a common diameter, but more or fewer than seven may be employed, as may rods 1030 of differing diameters) disposed within and encapsulated by the outer sleeve 1036. According to exemplary embodiments, the outer sleeve 1036 and the rods 1030 are all fabricated (at least in part) from a superelastic material, e.g., nickel titanium. The elongated member 1084 extends axially along an axis 1024, and one of the rods 1030 is disposed along the axis 1024 and extends beyond the ends 1066, 1068 of the axial portion 1062 of the outer sleeve 1036 and into the hollow areas defined by the end portions 1064 of the outer sleeve 1036 to an extent of the inner walls thereof The remaining rods 1030 are disposed radially around the axially-disposed rod 1030, and are shorter than the axially-disposed rod 1030 so as to accommodate the respective radial geometries of the end portions 1064. The cumulative transverse extent of the rods 1030 represent a substantial proportion of the inner diameter of the outer sleeve 1036, such that the shape and/or outer dimensions of the outer sleeve 1036 are substantially supported against crushing, plastic deformation, and/or galling, etc. At the same time, there exists radial space and/or spaces of a sufficient extent/s between and/or among the rods 1030, and between the rods 1030 and the outer sleeve 1036, so as to permit relative movement of such components relative to each other along the axial direction for purposes of allowing the elongated member 1084 to bend, flex and/or deflect along any and/or substantially all lateral/transverse directions.

Referring now to FIG. 14, an elongated member 1086 is illustrated that includes an outer sleeve 1088, and a series of structural elements 1090 disposed within and encapsulated by the outer sleeve 1036. The shell 1088 is substantially similar to the shell 1036 described and illustrated hereinabove with reference to FIG. 13, at least except insofar as the outer sleeve 1088 includes end caps 1092 that are flattened as compared to the end caps 1064 of the outer sleeve 1036, and/or do not necessarily exhibit the hemispheric-type shape thereof. The structural elements 1090 are 1) fabricated from a structurally rigid material, e.g., a steel that is compatible with (e.g., will not tend to induce galvanic corrosion in, and/or otherwise react with) the superelastic material of the outer sleeve 1088, 2) spherically shaped (e.g., with substantially identical diameters corresponding to and/or matched with an inner diameter of the outer sleeve 1088 so as to provide cylindrical shape support thereto), and 3) relatively tightly packed between the end caps 1092. The elongated member 1086 is substantially axially incompressible due to the tight packing of the structural elements 1090 between the end caps, and is substantially axially inextensible due to the tensile strength/rigidity of the outer sleeve 1088. The structural elements 1090 have substantially smooth outer surfaces, generally remain in point contact with each other, and are adapted (e.g., by virtue of their spherical shape) to rotate relative to/around each other without offering substantial resistance to such motion. Accordingly, such bending stiffness as is present in the elongated member 1086 is substantially solely based on the material and structural properties of the outer sleeve 1088. In this regard, it should be noted that without the shape/radial dimensional support provided to the outer sleeve 1088 by the structural elements 1090, the capacity of the outer sleeve 1088 to supply such bending stiffness would be reduced and/or substantially degraded.

Referring to FIG. 15, an elongated member 1094 is illustrated that includes an outer sleeve 1036 and a coil spring 1096 disposed within and encapsulated by the outer sleeve 1036. The elongated member 1084 extends axially along the axis 1024, and the coil spring 1096 is disposed along the axis 1024 and extends beyond the ends 1066, 1068 of the axial portion 1062 of the outer sleeve 1036 and into the hollow areas defined by the end portions 1064 of the outer sleeve 1036 to an extent of the inner walls thereof. The coil spring 1096 is fabricated from a structurally rigid material, e.g., a steel that is compatible with (e.g., will not tend to induce galvanic corrosion with, and/or otherwise react with) the superelastic material of the outer sleeve 1036, and is cylindrically shaped (e.g., with a diameter corresponding to and/or matched with an inner diameter of the outer sleeve 1036 so as to provide cylindrical shape support thereto). Bending stiffness of the elongated member 1094 is an additive function of the individual bending stiffnesses of outer sleeve 1036 (e.g., as supported by the coil spring 1096) and coil spring 1096.

By comparison, FIG. 16 illustrates an elongated member 1098 that is substantially similar to the elongated member 1094 of FIG. 15, at least except insofar as the outer sleeve 1100 thereof is fabricated, not from a superelastic material, but rather from a biocompatible polymer of suitable toughness and durability to permit the elongated member 1098 to interconnect with conventional spine attachment devices and/or the attachment members 22 (see FIGS. 1-3) thereof. Accordingly, such bending stiffness as is present in the elongated member 1098 is substantially solely based on the material and structural properties of the coil spring 1096 thereof. Referring again to FIG. 13, an alternative version (not specifically shown) of the elongated member 1084 illustrated in FIG. 13 and described hereinabove can be provided by substituting the outer sleeve 1100 of the elongated member 1094 of FIG. 16 for the outer sleeve 1036 of the elongated member 1084. In accordance with such construction, such bending stiffness as would be present in the alternative version of the elongated member 1084 would be substantially solely based on the number, material, and structural properties of the various rods 1030. A similar substitution may be made for the outer sleeve 36

Turning now to FIG. 17, an elongated member 2102 is illustrated that includes a coil spring 2104 and a cable 2106. The elongated member 2102 extends in an axial direction (e.g., along an axis 2024), insofar as the coil spring 2104 is axially aligned with (e.g., defines) the axis 2024, and the cable 2106 extends axially through the coil spring 2104, and is also axially aligned with the axis 2024. An outer diameter 2108 of the cable 2106 is of an extent compatible with an inner diameter 2110 of the coil spring 2104 such that an outer peripheral surface 2112 of the cable 2106 is substantially limited with respect to transverse movement relative to the coil spring 2104, and/or is positively prevented from so moving. The coil spring 2104 is ordinarily in a fully compressed state (e.g., when the elongated member 2102 is in a substantially straight and/or linear configuration), and when so compressed, renders the elongated member 2102 substantially incompressible as against axial forces arrayed in compression. The cable 2106 is of conventional construction (e.g., steel wire rope), and as such renders the elongated member 2102 substantially inextensible as against axial forces arrayed in tension. Both the coil spring 2104 and the cable 2106 can extend substantially the entire length of the elongated member 2102 and are either attached to each other (e.g., at one or more locations along the length of the elongated member 2102) or are attached in common to a third element of structure (not shown) such that relative motion between the coil spring 2104 and the cable 2106 along the axial direction is substantially reduced and/or prevented. The elongated member 2102 can include an outer sleeve (not specifically shown) such as one of the outer sleeves 1036, 1088 of FIGS. 13 and 14 respectively, or such as the outer sleeve 1100 of FIG. 16, wherein either or both the coil spring 2104 and the cable 2106 are partially and/or completely enveloped or encapsulated by such outer sleeve (not shown). In some such embodiments of the elongated member 2102, the cable 2106 is affixed to and/or protrudes slightly out of either or both ends of such outer sleeve (not shown), so as to permit purchase to be gained on the cable 2106 (e.g., so as to permit one or more of the attachment member 22 (FIGS. 1-3) to be attached directly thereto, thereby exploiting the substantial inextensibility of the cable 2106).

FIG. 18 illustrates an elongated member 3114 that includes an axially-extending rod 3030 and, at least in the embodiment illustrated in FIG. 18, includes no further structure. The rod 3030 is fabricated from a superelastic material, e.g., a nickel titanium alloy, and includes a substantially constant transverse diameter scaled in size such that the elongated member 3114 offers a predetermined stiffness against lateral/transverse bending.

In FIG. 19 is shown another elongated member 3116 consisting solely of a rod, e.g., a rod 3118. The rod 3118 is substantially similar to the rod 3030, at least except insofar as it includes an axial portion 3120 along which the transverse diameter of the rod 3118 is reduced, e.g., from a substantially constant, relatively larger diameter at two spaced-apart axial locations 3122, 3124 at opposite ends of the axial portion 3120, to a substantially constant, relatively smaller diameter along a substantial proportion of the axial length of the axial portion 3120. In operation, the rod 3118 can connect to spine attachment elements along the axial locations 3122, 3124. The overall bending stiffness of the elongated member 3116 can be tuned by selecting for the transverse diameter/dimension of the axial portion 3120 an appropriate/corresponding extent.

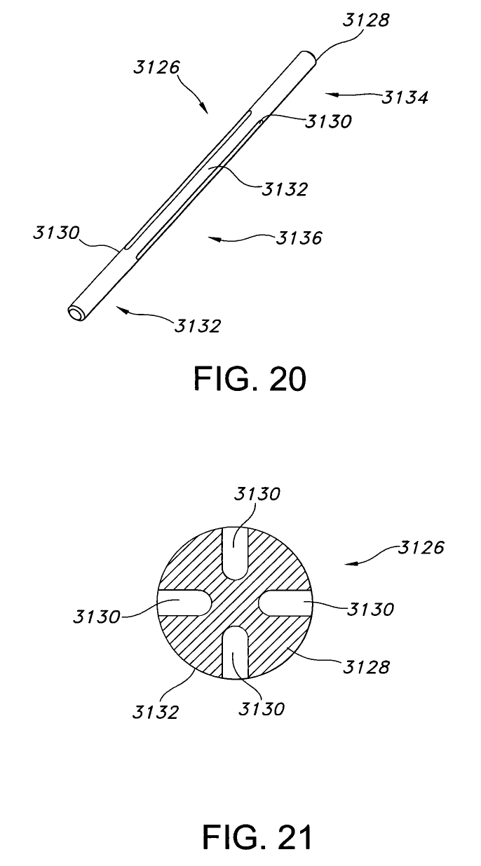

In FIGS. 20-21 is shown another elongated member 3126 consisting solely of a rod, e.g., a rod 3128. The rod 3128 is substantially similar to the rod 3030 of FIG. 18, at least except insofar as it includes longitudinal channels 3130 cut into and/or formed in the material of a peripheral outer surface 3132 of the rod 3128. The channels 3130 form a fluted configuration in which the channels 3130 are arranged in a regular array about the axial direction of extension of the rod (e.g., along the axis 3024). While four such channels 3130 are shown, more or fewer than four can be cut and/or formed in the rod 3128. In operation, the rod 3128 can connect to spine attachment elements along axial locations 3132, 3134 disposed at opposite ends of an axial portion 3136 of the rod 3128 in which the channels 3130 are formed. The overall bending stiffness of the elongated member 3126 can be tuned by altering the number, shape, and/or size of the channels 3130 as necessary/as desired.

Referring now to FIGS. 22-24, an elongated member 3134 is shown that includes a rod 3136, and, at least in the embodiment illustrated in FIGS. 22-24, includes no further structure. The rod 3136 extends in an axial direction (e.g., along an axis 3024), and is fabricated from a relatively structurally stiff, biocompatible metallic material, e.g., stainless steel, titanium or the like, and has a basic diameter 3138 that is substantially cylindrical. Cut into and/or formed in a peripheral outer surface 3140 of the rod 3136 are a first, second, third, and fourth axially-extending series 3142, 3144, 3146, 3148 of facets or channels 3150. The channels 3150 extend transversely straight across the material of the rod 3136 to a common radial depth or extent which is less than half that of the diameter 3138. The channels 3150 of the first and second series 3142, 3144 are formed on diametrically opposite sides of the axis 3024 from each other. The channels 3150 of the third and fourth series 3146, 3148 are also formed on diametrically opposite sides of the axis 3024 from each other, the transverse direction of extension of the channels 3150 of the third and fourth series 3146, 3148 being rotated ninety degrees relative to the transverse direction of extension of the channels 3150 of the first and second series 3142, 3144. Between each pair of opposing channels 3150 remains an axially-disposed extent 3152 of the material of the rod 3136 which is as wide as the rod 3136 in a first direction 3154, but which is relatively slender compared thereto in a second direction 3156 perpendicular to the first direction.

In operation, the rod 3136 can connect to spine attachment elements along axial locations 3158, 3160 disposed at opposite ends of an axial portion 3162 of the rod 3136 in which the channels 3150 are formed. Bending, flexure, and/or deflection of the rod 3136 is permitted substantially only at/along the numerous axially-disposed extents 3152 without risk of plastic deformation of the material of the rod 3136. By cutting/forming channels 3150 of an appropriate number and to an appropriate depth in the rod 3136, the overall bending stiffness of the rod 3136 can be reduced to a predetermined level. Because of the regular radial arrangement of the first, second, third, and fourth series 3142, 3144, 3146, 3148 of channels 3150, the flexibility produced thereby in the rod 3136 is substantially even as to any and/or all transverse directions of bending, flexure, and/or deflection.

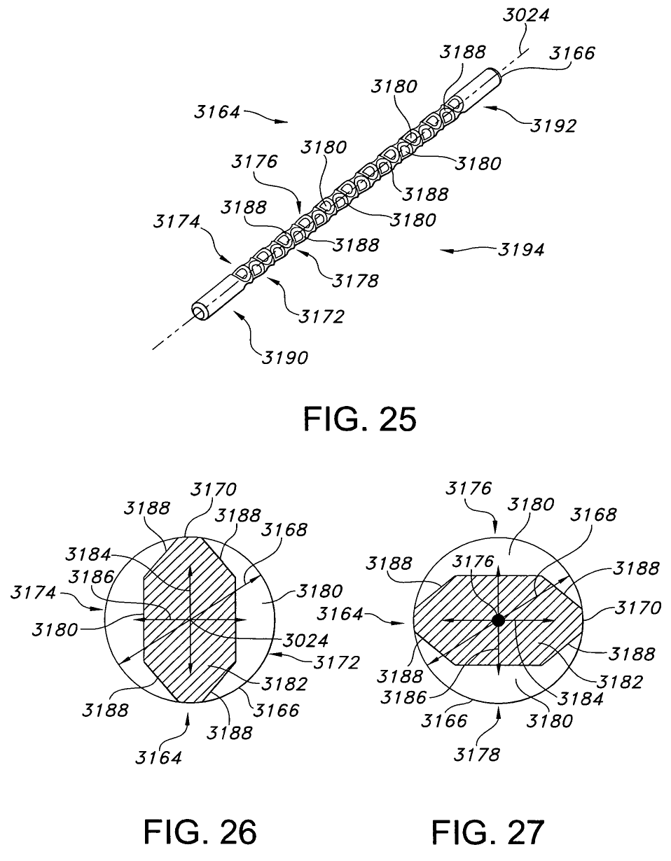

Referring now to FIGS. 25-27, an elongated member 3164 is shown that includes a rod 3166, and, at least in the embodiment illustrated in FIGS. 25-27, includes no further structure. The rod 3166 is substantially similar to the rod 3136 described above with reference to FIGS. 22-24, with differences as described hereinbelow. The rod 3166 extends in an axial direction (e.g., along an axis 3024), and has a basic diameter 3168 that is substantially cylindrical. Cut into and/or formed in a peripheral outer surface 3170 of the rod 3166 are a first, second, third, and fourth axially-extending series 3172, 3174, 3176, 3178 of facets or channels 3180. The channels 3180 extend transversely straight across the material of the rod 3166 to a common radial depth or extent which is less than half that of the diameter 3168, and which is less deep than the channels 3150 associated with the rod 3136 illustrated in FIGS. 22-24. The channels 3180 of the first and second series 3172, 3174 are formed on diametrically opposite sides of the axis 3024 from each other. The channels 3180 of the third and fourth series 3176, 3178 are also formed on diametrically opposite sides of the axis 3024 from each other, the transverse direction of extension of the channels 3180 of the third and fourth series 3176, 3178 being rotated ninety degrees relative to the transverse direction of extension of the channels 3180 of the first and second series 3172, 3174. Between each pair of opposing channels 3180 remains an axially-disposed extent 3182 of the material of the rod 3166 which is as wide as the rod 3166 along a first direction 3184, but which is to a certain extent less wide than the rod 3166 along a second direction 3186 perpendicular to the first direction 3184.

The channels 3180 are relatively wider than the channels 3150 (FIG. 22) and, as described above, shallower. Comers 3188 of all the channels 3180 are broken/beveled to a substantially greater extent than the channels 3150 (FIGS. 23-24) such that a portion of the material of the rod 3166 is removed at opposite diametrical ends of the axially disposed extents 3182. The extents 3182 are accordingly necked-down so as to be approximately as thick as the extents 3152 (FIGS. 23-24) at such diametrical ends.

In operation, the rod 3166 can connect to spine attachment elements along axial locations 3190, 3192 disposed at opposite ends of an axial portion 3194 of the rod 3166 in which the channels 3180 are formed. Bending, flexure, and/or deflection of the rod 3166 is permitted substantially only at/along the numerous axially-disposed extents 3182 without risk of plastic deformation of the material of the rod 3166. The relatively wider dimensions of the extents 3182 produce relatively less flexibility in the rod 3166 than the relatively narrower dimensions 3152 produce in the rod 3136 (FIGS. 22-24), as may be desired and/or necessary in certain applications. The broken corners 3188 of the channels 3180 smooth the contours of the rod 3166 so as to ensure that the rod 3166 manifests substantially the same flexibility in any and/or substantially all transverse directions, and not just in the two perpendicular transverse directions defined by the four series of channels 3180. (As will be apparent to those of skill in the art in light of the present disclosure, similarly large sized broken corners are not required in the context of the relatively more flexible rod 3136 (FIG. 22).)

It will be understood that the embodiments of the present disclosure are merely exemplary and that a person skilled in the art may make many variations and modifications without departing from the spirit and scope of the invention. All such variations and modifications, including those discussed above, are therefore intended to be included within the scope of the present invention as described by the following claims appended hereto.

1. An elongated member configured and dimensioned for implantation adjacent the spine of a patient such that an axial span of said elongated member extends in an axial direction across at least one spinal level thereof and is adapted to promote spinal stabilization across said at least one spinal level, said axial span further manifesting a radially segmented geometry relative to said axial direction.

2. An elongated member according to claim 1, wherein said elongated member is configured and dimensioned for implantation adjacent the spine of the patient such that at least two axial spans of said elongated member extend in respective axial directions across respective spinal levels thereof and are adapted to promote spinal stabilization across said spinal levels, each axial span of said at least two axial spans manifesting a radially segmented geometry relative to said axial direction.

3. An elongated member according to claim 1, wherein said axial span has a rod-like profile, and is adapted to be coupled to said spine of said patient.

4. An elongated member according to claim 3, wherein said rod-like profile of said axial span includes a diameter in a range from about 5.5 mm to about 6.35 mm.

5. An elongated member according to claim 1, wherein said axial span is adapted to permit a spinal mounting structure to attach to said elongated member at multiple points along a length of said axial span so as to accommodate a range of different patient anatomies and intervertebral heights.

6. An elongated member according to claim 1, wherein said axial span is substantially rigid as against axial forces arrayed in compression.

7. An elongated member according to claim 1, wherein said axial span is substantially rigid as against axial forces arrayed in tension.

8. An elongated member according to claim 1, wherein said radially segmented geometry of said axial span permits said axial span to bend along any and substantially all transverse directions while promoting efficacious spinal stabilization across said spinal level during at least one of spinal flexion, spinal extension, spinal lateral bending, and axial rotation.

9. An elongated member according to claim 1, wherein said axial span is adapted to provide efficacious spinal stabilization across said spinal level during spinal flexion in which said spinal level defines an anterior bend of at least approximately three to seven degrees.

10. An elongated member according to claim 1, wherein said axial span is adapted to provide efficacious spinal stabilization across said spinal level during spinal extension in which said spinal level defines a posterior bend of at least approximately three to seven degrees.

11. An elongated member according to claim 1, wherein said axial span is adapted to provide efficacious spinal stabilization across said spinal level during spinal bending in which said spinal level defines a lateral bend of at least approximately three to seven degrees.

12. An elongated member according to claim 1, wherein said radially segmented geometry includes a rod of radially unitary construction and extending in said axial direction, and at least one sleeve extending in said axial direction and surrounding said rod.

13. An elongated member according to claim 12, wherein at least one of said rod and said sleeve is fabricated from a superelastic material.

14. A surgically implantable spinal support rod having an axial span extending in an axial direction so as to span at least one spinal level, said axial span manifesting a radially segmented geometry relative to said axial direction.

15. A spinal support rod according to claim 14, wherein said radially segmented geometry manifested by said axial span includes at least one pair of axially-extending adjacent surfaces adapted to move relative to each other along said axial direction during a transverse deflection of said axial span.

16. A spinal support rod according to claim 15, wherein a pair of said at least one pair of axially-extending surfaces includes a first substantially cylindrically shaped surface and a second substantially cylindrically shaped surface.

17. A spinal support rod according to claim 16, wherein each of said first and second substantially cylindrically shaped surfaces faces radially outward toward the other of said first and second substantially cylindrically shaped surfaces.

18. A spinal support rod according to claim 16, wherein said first substantially cylindrically shaped surface and said second substantially cylindrically shaped surface are substantially axially aligned with respect to each other.

19. A spinal support rod according to claim 14, wherein said axial span has a rod-like profile, and is adapted to be coupled to said spine of said patient via attachment to spine attachment devices configured for coupling conventional support rods to said spine.

20. An elongated member according to claim 19, wherein said rod-like profile of said axial span includes a diameter in a range of from about 5.5 mm to about 6.35 mm.

21. A kit for assembling a dynamic spinal support system, comprising:
a spinal support rod having an axial span extending in an axial direction so as to span at least one spinal level, said axial span manifesting a radially segmented geometry relative to said axial direction; and
a plurality of spine attachment devices attachable to said axial span so as to couple said spinal support rod to the spine of a patient across said spinal level.

22. A kit for assembling a dynamic spinal support system according to claim 20, wherein at least one of said spine attachment devices is selected from the group consisting of a pedicle screw, a hook, a mounting plate and a stem.

23. An elongated member configured and dimensioned for implantation adjacent the spine of a patient such that an axial span of said elongated member extends in an axial direction across at least one spinal level thereof and is adapted to promote efficacious spinal stabilization across said at least one spinal level, said axial span including a sleeve and a series of structural members aligned along said axial direction, enclosed within said--sleeve, and adapted to support said sleeve against lateral buckling when said sleeve includes a lateral bend and is supporting said spine across said spinal level.

24. An elongated member according to claim 23, wherein said sleeve is adapted to generate an internal spring force in opposition to said lateral bend as said sleeve deflects so as to form said lateral bend.

25. An elongated member according to claim 24, wherein said sleeve is fabricated from a superelastic material.

26. An elongated member according to claim 25, wherein said superelastic material is an alloy of titanium.

27. An elongated member according to claim 23, wherein said structural members are substantially spherical in shape.

28. An elongated member according to claim 27 wherein said sleeve is substantially cylindrical in shape.

29. An elongated member configured and dimensioned for implantation adjacent the spine of a patient such that an axial span of said elongated member extends in an axial direction across at least one spinal level thereof and is adapted to promote efficacious spinal stabilization across said at least one spinal level, said axial span including an axial sleeve, and a coil spring disposed within said axial sleeve.

30. An elongated member according to claim 29, wherein said axial sleeve is fabricated from a superelastic material.

31. An elongated member according to claim 29, wherein said axial sleeve is fabricated from an alloy of titanium.

32. An elongated member according to claim 29, wherein said axial sleeve is fabricated from a polymeric material.

33. An elongated member according to claim 29, wherein said coil spring is sized and oriented so as to support a peripheral shape of said axial sleeve against at least one of crushing and buckling during said spinal stabilization.

34. An elongated member configured and dimensioned for implantation adjacent the spine of a patient such that an axial span of said elongated member extends in an axial direction across at least one spinal level thereof and is adapted to promote efficacious spinal stabilization across said at least one spinal level, said axial span including an axially-extending coil spring, and a restraining element disposed within said coil spring and extending at least partially through said coil spring in said axial direction so as to limit an axial extension of said elongated member.

35. An elongated member according to claim 34, wherein said restraining element includes a cable adapted to render said elongated member substantially rigid as against axial forces arrayed in compression.

36. An elongated member according to claim 35, wherein said cable is a wire rope cable.

37. A spinal support bar configured and dimensioned for implantation adjacent the spine of a patient such that said spinal support bar extends in an axial direction across at least one spinal level thereof and is adapted to promote efficacious spinal stabilization across said at least one spinal level, said spinal support bar being of unitary construction, both along said axial direction, and radially relative to said axial direction, and said spinal support bar being further adapted to deflect laterally so as to permit at least three to seven degrees of bending in said spine across said spinal level in at least one of spinal flexion, spinal extension, and spinal lateral bending.

38. A spinal support bar according to claim 37, wherein said spinal support bar manifests a substantially constant cross-sectional geometry across said spinal level.

39. A spinal support bar according to claim 38, wherein said cross-sectional geometry defines a circle.

40. A spinal support bar according to claim 37, wherein said spinal support bar includes a central span extending in said axial direction, and channels formed in said central span so as to increase a transverse flexibility of said central span.

41. A spinal support bar according to claim 40, wherein said channels extend in said axial direction.

42. A spinal support bar according to claim 40, wherein said channels extend transversely relative to said axial direction.

43. A spinal support bar according to claim 37, wherein said spinal support bar includes a central span, a first end span, and a second end span disposed opposite said central span from said first end span, said central span being associated with a cross-section of a reduced area relative to respective cross-sections of said first and second end spans.

44. An elongated member according to claim 43, wherein said central span is associated with a circular cross-section of a reduced diameter relative to respective circular cross-sections of said first and second end spans.
